(19) United States
(12) Patent Application Publication
(10) Pub. No.: US 2025/0125009 A1
(43) Pub. Date: Apr. 17, 2025

(54) Multistep Diagnostic Methods using Hand-Held PCR

(71) Applicant: Nant Holdings IP, LLC, Culver City, CA (US)

(72) Inventor: Patrick Soon-Shiong, Culver City, CA (US)

(73) Assignee: Nant Holdings IP, LLC, Culver City, CA (US)

(21) Appl. No.: 18/918,679

(22) Filed: Oct. 17, 2024

Related U.S. Application Data
(60) Provisional application No. 63/601,127, filed on Nov. 20, 2023.
(60) Provisional application No. 63/591,045, filed on Oct. 17, 2023.

(51) Int. Cl. G16B 25/10; C12Q 1/6844; C12Q 1/6888; G16H 20/10; G16H 50/20

(52) U.S. Cl. CPC G16B 25/10; C12Q 1/6844; C12Q 1/6888; G16H 20/10; G16H 50/20; C12Q 2600/158

(57) ABSTRACT

Computer-based systems and methods are presented in which digital data from a subject are used to provide an initial diagnosis, and in which a polymerase chain reaction (PCR)-based assay is performed at the point-of-care to confirm and refine the initial diagnosis. Advantageously, the polymerase chain reaction (PCR)-based assay is configured as a hand-held portable device and uses a plurality of nucleic acid primers that are chosen as a function of the initial diagnosis. The primers are further chosen to allow for refinement of the initial diagnosis to enable generation of a treatment plan and assist in appropriate treatment at the point of care or use.

This application claims priority to our copending US Provisional patent applications with the Ser. No. 63/591,045, filed Oct. 17, 2023, and the Ser. No. 63/601,127, filed Nov. 20, 2023, both of which are incorporated by reference herein.

### FIELD OF THE INVENTION

The field of the invention is devices and methods to confirm and refine an initial finding that is based on digital data using further digital data from a point-of-test or point-of-care device, and especially as it relates to hand-held PCR devices.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Differential diagnostic methods have been employed for a long time and are typically performed at a physician's office, often after a physician reviewing a patient's chart and/or test data from blood and/or radiographic tests. More recently, with the advent of computer-assisted medicine, patient data can be evaluated using computer algorithms to suggest likely conditions, and in some cases also treatment options. Indeed, where genomic or transcriptomic tests are performed, computer algorithms are indispensable to process the vast quantity of data. However, such analyses will often not make use of prior existing patient records or lack depth of analysis or situational awareness to enable determination of appropriate treatment options. For example, where a long-term diabetic patient presents with a diabetic foot ulcer that was previously and unsuccessfully treated with one antibiotic, image analysis alone or other existing digital data will not inform a physician of proper treatment options. As such, the wrong therapeutic agent may be administered, thereby prolonging patient risk and pain, and delaying proper care. On the other hand, where a physician evaluates the diabetic lesion at the point of care, in all or almost all cases no immediate analysis is performed, and subsequent tests must be ordered to properly diagnose the condition. These and other difficulties may be further exacerbated where a patient changes the medical care provider and records have not been provided by the patient to the new provider. In almost all of such cases, patient history is only available in electronic format and in an often incomplete narrative of the patient. This is particularly true where a prior medical record includes a large number of clinical data.

Thus, even though various systems and methods for medical diagnoses are known in the art, all or almost all of them suffer from several drawbacks. Therefore, there remains a need for compositions and methods for improved diagnostic methods, particularly where such methods enable point-of-care or point-of-use tests that enable accurate diagnosis and treatment.

### SUMMARY OF THE INVENTION

The inventive subject matter is directed to various computer-based systems and methods in which digital data from a subject are used to provide an initial diagnosis, and in which a polymerase chain reaction (PCR)-based assay is performed at the point-of use or point-of-care to confirm and refine the initial diagnosis. Advantageously, the polymerase chain reaction (PCR)-based assay uses a plurality of nucleic acid primers that are chosen as a function of the initial diagnosis, and where the primers are further chosen to allow for refinement of the initial diagnosis to enable generation of a treatment plan and assist in appropriate treatment at the point of care or use.

In one aspect of the inventive subject matter, the inventor contemplates a computer assisted method of translating diagnostic data into polymerase chain reaction (PCR)-based clinically actionable patient data that includes the steps of (a) obtaining, via at least one processor, digital diagnostic data from a patient; (b) processing the digital diagnostic data via execution of an implementation of an algorithm, wherein the algorithm probabilistically maps the patient digital diagnostic data into a first diagnosis; (c) profiling a biological sample from the patient on a hand-held portable PCR array by determining expression levels of clinically significant genes, wherein the PCR array comprises unique nucleic acid primers for evaluation of specific expression of multiple genes relevant to the first diagnosis; (d) mapping, via the at least one processor, the expression levels to a genetically informed second diagnosis for the patient; and (c) providing the genetically informed second diagnosis for the patient to a computer device.

Most typically, the first diagnosis will relate to an infectious disease and may therefore relate to a bacterial, viral, bacterial, fungal, or parasitic infection. For example, the first diagnosis may relate to a urinary tract infection or a wound infection. In further examples, the first diagnosis may also relate to resistance to treatment of an infectious disease, such as resistance to treatment of a bacterial, viral, bacterial, fungal, or parasitic infection. Thus, the first diagnosis may also relate to resistance to treatment with antibiotics. In still further examples, the first diagnosis may relate to a cancer and/or may comprise a determination of a cancer type (e.g., via a theranostic procedure).

Depending on the type of diagnosis, the digital diagnostic data may be processed to arrive at the first diagnosis via a digital pathology platform. Most typically, the algorithm for processing the digital diagnostic data may comprise a machine learning algorithm or a reasoning algorithm executed by a reasoning engine. Therefore, contemplated digital diagnostic data may comprise whole genomic data and/or transcriptomic sequencing data, or data are derived from culturing a biological sample (e.g., urine sample, blood sample, respiratory tract sample, mucosal sample, or tissue biopsy) of the patient. In other embodiments, the digital diagnostic data may be derived from a radiological image.

It is also contemplated that such method may include a step of generating a treatment plan at a point of care based on the second diagnosis, and most typically also a step of administering a drug (e.g., antibiotic or a chemotherapeutic drug) based on the treatment plan.

In another aspect of the inventive subject matter, the inventor also contemplates a computer assisted method of translating polymerase chain reaction (PCR)-based diagnostic data into clinically actionable patient data that includes the steps of (a) obtaining, via at least one processor, digital PCR-based diagnostic data from a patient, wherein the PCR-based diagnostic data comprise respective nucleic acid expression levels for a plurality of genes in a gene array, and wherein a selection of the genes in the array is determined by a preliminary first diagnosis; (b) processing the digital PCR-based diagnostic data via execution of an implementation of an algorithm, wherein the algorithm probabilistically maps the digital PCT-based diagnostic data into a second diagnosis; (c) mapping, via the at least one processor and using the second diagnosis, the digital PCR-based diagnostic data to at least a machine-learning or artificial intelligence (AI) informed third diagnosis for the patient; and (d) providing the machine-learning or AI informed third diagnosis for the patient to a computer device.

In preferred embodiments, the PCR-based digital data may be obtained from profiling a biological sample from the patient on a hand-held portable PCR array, and/or the preliminary first diagnosis is derived from digital histopathology. The third diagnosis may then be derived from a reasoning engine. In further embodiments, the preliminary first diagnosis may be derived from a radiological image, or from culturing a biological sample (e.g., urine sample, blood sample, respiratory tract sample, mucosal sample, or tissue biopsy) of the patient. Additionally, it is contemplated that the preliminary first diagnosis is derived from whole genomic or transcriptomic sequencing, and/or that the second and/or third diagnosis comprises a determination of antibiotic resistance by the patient or a determination of chemotherapeutic resistance by the patient, or a determination of anti-fungal resistance by the patient. In still further embodiments, the preliminary first diagnosis may comprise a determination of a cancer type (e.g., via a theranostic procedure).

In further contemplated embodiments, the method may also include a step of generating a treatment plan at a point of care based on the third diagnosis. Typically, but not necessarily, the digital PCR-based diagnostic data is processed to form the second diagnosis by a digital pathology platform, and/or the algorithm that processes the digital PCR-based diagnostic data algorithm may comprise a machine learning algorithm or a reasoning algorithm executed by a reasoning engine. In still further aspects of contemplated methods, the plurality of genes in the gene array may be selected from the group consisting of genes associated with cancer, genes associated with viral infection, genes associated with bacterial infection, genes associated with fungal infection, genes associated with parasitic infection, and genes associated with cardiac pathology.

In yet another aspect of the inventive subject matter, the inventor contemplates a computer assisted method of translating water or food testing data into polymerase chain reaction (PCR)-based actionable data that includes the steps of (a) obtaining, via at least one processor, digital data from an assay selected from the group consisting of a food quality assay, a beverage quality assay, and a water quality assay, wherein the assay determines a presence and/or a level of at least one biological contaminant in a sample, and wherein the assay is performed on one portion of the sample; (b) processing the digital data via execution of an implementation of an algorithm, wherein the algorithm probabilistically maps the data into a first determination of biological contamination; (c) profiling another portion of the sample on a hand-held portable PCR array to determine expression levels of genes related to microbial presence and quantity, wherein the PCR array comprises multiple unique nucleic acid primers for evaluation of expression of respective multiple genes relevant to the first determination; (d) mapping, via the at least one processor, the gene expression levels to a genetically informed and actionable second determination; and (c) providing the genetically informed and actionable second determination of biological contamination to a computer device.

In some embodiments, the biological contaminant may be a bacterium, a virus, a yeast, a fungus, a microbe, or a parasite. For example, the biological contaminant may be a Salmonella spec. Therefore, the PCR array may comprise nucleic acid primers specific for Salmonella enterica or Salmonella bongori serotypes. While other types of assays are also deemed suitable, especially contemplated assays include Loop-mediated isothermal amplification (LAMP), Recombinase Polymerase Amplification (RPA), Recombinase Aided Amplification (RAA), Rolling Circle Amplification (RCA), and Saltatory Rolling Circle Amplification (SRCA).

Such methods may be particularly suitable for use in in-patient and/or outpatient medical facilities (e.g., in a nursing home). Alternatively, contemplated methods may be suitable for use in a cruise ship or a passenger airplane, or in a food preparation facility (e.g., in a kitchen, a restaurant, or a cafeteria). As will be readily appreciated, such methods may additionally include a step of applying a decontamination protocol to an object based on the second determination, wherein the sample was obtained from the object.

In a further aspect of the inventive subject matter, the inventor contemplates computer assisted method of translating diagnostic data into polymerase chain reaction (PCR)-based actionable non-human subject data that includes the steps of (a) obtaining, via at least one processor, digital diagnostic data from a non-human subject; (b) processing the digital diagnostic data via execution of an implementation of an algorithm, wherein the algorithm probabilistically maps the digital diagnostic data into a first diagnosis; (c) profiling a biological sample from the non-human subject on a hand-held portable PCR array by determining expression levels of nutritionally significant genes, wherein the PCR array comprises multiple unique nucleic acid primers for evaluation of expression of respective multiple genes relevant to the first diagnosis; (d) mapping, via the at least one processor, the expression levels to a genetically informed second diagnosis for the subject; and (e) providing the genetically informed second diagnosis for the subject to a computer device.

In an embodiment, the first diagnosis may relate to a state of nutritional deficiency in the subject, such as a macronutrient deficiency (e.g., protein, fat, and/or carbohydrate deficiency) or a micronutrient deficiency (e.g., vitamin or mineral deficiency) in the subject. In further embodiments, the digital diagnostic data may be processed to arrive at the first diagnosis via a digital pathology platform, and/or the algorithm may comprise a machine learning algorithm or a reasoning algorithm executed by a reasoning engine.

Most typically, but not necessarily, the first diagnosis may be derived from whole genomic or transcriptomic sequencing, and/or may be derived from culturing a biological sample of the patient. For example, suitable biological samples include a urine sample, a blood sample, a respiratory tract sample, a mucosal sample, or a tissue biopsy. Alternatively, the first diagnosis may also be derived from a radiological image.

Preferably, such methods will further include a step of generating a treatment plan at a point of care based on the second diagnosis, and/or a step of generating a nutrition plan at a point of care based on the second diagnosis. Thus, contemplated methods will also comprise a step of administering a treatment or a nutritional supplement to the non-human subject based on the second diagnosis and treatment plan or nutrition plan.

Various objects, features, aspects and advantages of the inventive subject matter will become more apparent from the following detailed description of preferred embodiments.

### DETAILED DESCRIPTION

The inventor has now discovered that prior diagnoses can be readily confirmed and refined using multistep diagnostic devices, systems, and methods that will take advantage of a point-of-care device (e.g., a portable/hand-held PCR array) that is selected or assembled based on prior known digital diagnostic data, and that generates PCR-based diagnostic data (typically at the point-of-care or point-of-use) that are then mapped to a genetically informed diagnosis that confirms, and preferably also refines a prior diagnosis using the PCR-based diagnostic data.

In this context, it should be especially appreciated that contemplated systems and methods are not limited to the provision of a prior diagnosis, but that an initial diagnosis can be generated from previously obtained digital diagnostic data. In such case, it is typically preferred that the previously obtained digital diagnostic data will be probabilistically mapped by a computer algorithm to so generate a first or initial diagnosis. As such, potential bias or even an incorrect diagnosis by a prior medical practitioner can be avoided.

Upon determination of the first or initial diagnosis, a PCR array is generated, or a premanufactured PCR array is selected in which the PCR array comprises unique nucleic acid primers for evaluation of specific expression of multiple genes relevant to the first diagnosis. As will be readily appreciated, the choice of the multiple genes may include genes that are found in the patient (typically human or other non-human mammalian subjects) and that are affected in their expression by the condition identified in the first or initial diagnosis. Alternatively, or additionally, the genes may also be part of a pathogen that is the etiological agent for the condition identified in the first or initial diagnosis. Therefore, and viewed from a different perspective, it should be appreciated that the PCR array will not only be used to query or identify gene expression profiles that are associated with the condition to so confirm the computer-generated diagnosis but can also be used to further qualify or refine the diagnosis to so generate a genetically informed second diagnosis.

In one exemplary use of contemplated systems and methods, a patient may present to a physician as a new patient. In this case, the patient had a documented history of a diabetic foot ulcer infection that was previously unsuccessfully treated with an antibiotic. The patient's history, including vital data, prior diagnoses, current and past medication and doses, and complete blood count and blood chemistry are digitally stored at the patient's insurance provider and medical record storage facility.

Upon examination of the patient's ulcer and suspicion of infection, the physician uses his point-of-care analytic device to request and receive the existing digital diagnostic data from the patient. The device then uses a microprocessor to process the digital diagnostic data via execution of an implementation of an algorithm, wherein the algorithm probabilistically maps the digital diagnostic data into a first diagnosis, which in this example, would be an initial diagnosis of an antibiotic-resistant wound infection. Most typically, such analysis can be performed locally on the device, or at least partially remotely on a networked device that informationally cooperates with the point-of-care analytic device to perform the probabilistic mapping. In most typical embodiments, the physician has a mobile device that connects to the point-of-care analytic device and the point-of-care analytic device provides the mobile device with a recommended PCR array test. Of course, it should be appreciated that the point-of-care analytic device may also directly provide such recommendation.

Regardless of the particular mechanism, it should be noted that a suitable PCR array test can be recommended only on the basis of existing medical records. Moreover, it should be appreciated that the selection of the PCR array may be entirely derived from the existing digital diagnostic data from the patient, typically by identifying an underlying etiology (e.g., metabolic or genetic disease, cancer, progressive organ failure, etc.) or causative agent (e.g., bacterial or viral infection, parasitic infestation, etc.) associated with the initial diagnosis. This identified etiology or causative agent will then inform the proper choice of nucleic acids for the PCR test. For example, where causative agents are to be detected, DNA-based or rRNA based PCR may be performed. On the other hand, rtPCR and/or qPCR may be performed where gene expression levels are particularly informative for a second diagnosis, for example, to identify expression of cell surface markers and/or chemotherapy resistance markers in cancer.

To confirm the initial diagnosis and to even provide a refined diagnosis, the physician can then perform a PCR array test after taking a sample of the infected wound, typically via a swab or biopsy punch. The so collected sample, is then placed into a sample or lysis buffer to liberate sufficient quantities of nucleic acids for detection. In this context, it should be appreciated that the nucleic acids for the subsequent PCR test in this particular example will be nucleic acids of pathogenic bacteria. As such, the PCR array will contain a variety of nucleic acid primers that are specific to the most common gram-positive and gram-negative bacteria found in wounds, including Staphylococcus aureus, Enterococcus faecalis, Enterococcus faecium, Escherichia coli (E. coli), Klebsiella oxytoca (KO), Enterobacter spp., Proteus mirabilis, Acinetobacter baumannii, and Pseudomonas aeruginosa. Moreover, the PCR array will also preferably comprise additional nucleic acid primers that can identify the presence of resistance genes such as mecA, mecC, vanA, vanB, among other suitable primers.

In a similar manner, numerous alternative PCR arrays are contemplated that can not only quickly and specifically identify a disease, condition, or infection, but also provide more detailed, treatment relevant, and actionable information about potential treatment resistance, likely positive or negative treatment response, sub-typing of cancer, etc. Such PCR arrays will in most cases be based on currently available molecular diagnostic markers associated with a disease or disorder. However, in other embodiments, diagnostic markers may also be identified in clinical tests, animal models, and cell-based models. In addition, it should be appreciated that the PCR arrays contemplated herein will include one or more positive and negative control primers, with a positive control primer typically targeting a ubiquitous human gene with known expression such as RNaseP or other suitable marker(s). Additionally, it should be appreciated that the PCR assay may be used as a qualitative assay or as a quantitative assay. As such, it should be noted that the PCR reaction may be a simple amplification method as presented above, or a quantitative PCR reaction such as a qPCT/rtPCR. In still further preferred aspects, the PCR array will be configured as a hand-held and easily portable unit that includes all reagents need to perform a (quantitative) PCR. There are numerous portable lab-on-a-chip devices known in the art (see e.g., NantNudge device), and all of those are deemed suitable for use herein.

Returning to the ulcer example and upon conclusion of the PCR assay, the digital PCRbased diagnostic data can then be processed either on the point-of-care analytic device, and more typically on an informationally coupled computing device that maps, via a processor, the expression levels or expression data to a genetically informed second diagnosis for the patient. In this example, the genetically informed second diagnosis for the patient may now inform the clinician that the ulcer is infected with Staphylococcus aureus carrying the mecA resistance gene. In this particular case, the physician is now apprised of a treatment option such as use of vancomycin. However, if the digital patient data also indicated renal impairment, alternate treatment options may be chosen, such as seftaroline or linczolid.

Therefore, it should be appreciated that the genetically informed second diagnosis for the patient may not only be a confirmation of the first or initial diagnosis, but may also include additional information (e.g., allergy relevant information, information relevant to avoidance or use of specific medication, etc.) that was obtained from the digital diagnostic data from the patient. Moreover, in most cases, the genetically informed second diagnosis will also refine the initial diagnosis and as such guide a physician to a more appropriate treatment. Viewed from a different perspective, and especially where gene expression data are obtained from a patient via the PCR assay, the patient's individual physiological situation may be taken into account to so increase likelihood of treatment success. Thus, contemplated systems and methods will render treatment more patient specific and so potentially more effective.

Preferably, but not necessarily, the point-of-care analytic device (or computing device informationally coupled to the point-of-care analytic device) may transmit the genetically informed second diagnosis to another computer such as a mobile device or tablet used by the treating physician. In addition, the point-of-care analytic device (or computing device informationally coupled to the point-of-care analytic device) may also generate a treatment recommendation or plan based on the second diagnosis, and the physician can subsequently treat the patient according to the treatment recommendation or plan (e.g., using a suitable antibiotic or chemotherapeutic drug.

With respect to the digital diagnostic data from the patient, it should be appreciated that the data may be in a variety of data formats. However, especially contemplated data formats will be data formats generally recognized and/or adopted in routine practice. Therefore, the data may be formatted following standards published by the Society for Clinical Data Management (SCDM) under the Good Clinical Data Management Practices (GCDMP) guidelines. For example, suitable data formats will thus include the Clinical Data Acquisition Standards Harmonization (CDASH) standard, the Study Data Tabulation Model Implementation Guide for Human Clinical Trials (SDTMIG) standard, or the Clinical Data Interchange Standards Consortium (CDISC) standard. As will be readily appreciated, all digital diagnostic data from the patient will be stored and/or transmitted in encrypted format to comply with relevant patient data privacy regulations.

It is also noted that the digital diagnostic data from the patient will not be limited to a single health issue but may in fact comprise a comprehensive patient data record. Accordingly, it is contemplated that the digital diagnostic data from the patient will include systemic data such as age, weight, height, vital statistics, etc., as well as results from gross examination, clinical lab data, imaging data (e.g., radiological), digital histopathology data, theranostic data. Moreover, and where available, the digital diagnostic data from the patient may also include various omics data, and especially whole genome data, exome sequencing data, transcriptome data, proteome data, SNP data, etc. Similarly, contemplated digital diagnostic data from the patient may also include additional molecular data such as cell surface markers (e.g., associated with cancer type, receptor status, HLA data, etc). In addition, contemplated digital diagnostic data from the patient will also include prior treatment data and prior treatment outcome data, particularly as it relates to pharmaceutical treatments and outcomes for such treatment (e.g., resistance to treatment with an antibiotic or relapse of a cancer after treatment with one or more chemotherapeutic drugs, etc.), allergy information, vaccination status, etc.

Consequently, it should be recognized that the digital diagnostic data from the patient may be obtained in a variety of manners. For example, the digital diagnostic data from the patient may be obtained from a patient sample such as a urine sample, a blood sample, a respiratory tract sample, a mucosal sample, or tissue biopsy, or may be derived from culturing a biological sample of the patient. Alternatively, or additionally, the digital diagnostic data from the patient may also be obtained from an X-ray or a scanning procedure (e.g., CT scan, MRI scan, PET scan, etc.) or a digital histopathology platform, or from a sequencing platform.

Depending on the particular digital diagnostic data from the patient, the first diagnosis (which may be an initial or first or preliminary diagnosis) may vary considerably. For example, where the data include imaging data, histopathology data, and/or omics data, contemplated diagnoses will typically be a diagnosis of a cancer or a cancer (sub) type. On the other hand, where the data are obtained from a blood draw and/or a culture of a sample, contemplated diagnoses will typically be a diagnosis of an infection or infectious disease such as a wound infection, a urinary tract infection, etc., which may be of bacterial, viral, fungal, or parasitic origin. As will be readily appreciated, such diagnosis may be further based on or make use of the ICD-10 classification to facilitate computational analysis, which will typically be performed using a reasoning engine, a machine learning algorithm, and/or probabilistic mapping. Thus, it should be noted that the digital diagnostic data may be mapped to a first or initial diagnosis entirely in silico. However, human intervention or guidance to arrive at the first or initial diagnosis is also deemed suitable.

There are various systems and methods known in the art that can associate the digital diagnostic data with an initial or first or preliminary diagnosis, and exemplary systems and methods using reasoning engines are described, in U.S. Pat. Nos. 9,530,100, 9,262,719, 10,255,552, 10,296,840, 10,762,433, and 10,296,839, all incorporated by reference herein. Where appropriate, contemplated algorithms may also include interference engines as described, for example, in U.S. Pat. No. 9,576,242, also incorporated by reference herein. As should be further appreciated, contemplated algorithms may be executed on one or more processors in the point-of-care analytic device and/or executed on one or more remote processors that is/are informationally coupled to the point-of-care analytic device.

Regardless of the manner of mapping the digital diagnostic data to a first diagnosis, it is contemplated that the first diagnosis is communicated to the medical professional (e.g., via a display of the point-of-care analytic device or via a separate computer that is informationally coupled to the point-of-care analytic device) for human review. In addition, the first diagnosis will typically also include information with regard to unique nucleic acid primers for a PCR reaction, wherein the information may comprise actual nucleic acid sequences, or codes for such sequences, or a code or instruction to use a specific PCR array containing sequences suitable for point-of-care analysis. As will be readily appreciated, the first diagnosis (or first determination of a biological contamination) will determine the appropriate choice of nucleic acid sequences for a subsequent PCR reaction. For example, where the presence of a pathogen is to be confirmed, nucleic acid primers may have a sequence that targets unique DNA or RNA (and especially rRNA) sequences of the pathogen for qualitative detection. Such detection may also include detection of one or more genes associated with treatment resistance such as antibiotic resistance genes. In another example, where susceptibility of a cancer cell to a chemotherapeutic drug is to be determined, nucleic acid primers will have a sequence targeting genes and/or RNA known to be prognostic for treatment resistance of success. Viewed from a different perspective, it should be appreciated that the PCR reaction may be a qualitative PCR, or a quantitative PCR (which may, for example, be an rtPCT or a qPCR).

In still further contemplated aspects, it is generally preferred that a physician will use a portable and/or hand-held PCR array that contains the nucleic acid primers for the PCR reaction. Most typically, but not necessarily, the hand-held PCR array will include an array of various nucleic acids related to the first diagnosis (or first determination of a biological contamination) to confirm the first diagnosis, and most preferably also to refine the diagnosis. For example, where the first diagnosis is a suspected infection, the array of nucleic acids may comprise primers to identify one or multiple different possible pathogens commonly found in wound infections as noted above. In another example, where the first diagnosis is a suspected cancer type, the array of nucleic acids may comprise primers to identify a cancer sub-type that may be characterized by the presence of absence of a cell surface marker (e.g., HER2, estrogen receptor, progesterone receptor) and/or by susceptibility to chemotherapeutic treatment on the basis of gene expression of genes attributed to the susceptibility.

Most typically, but not necessarily, contemplated portable and/or hand-held PCR arrays will include a preselected set of nucleic acid primers matching common first diagnoses, and will also include all reagents needed to perform the PCR reaction. Such reagents may also include sample preparation buffers such as lysis buffers to isolate or liberate circulating or cell free nucleic acids as are described, for example in U.S. Pat. Nos. 11,168,323, 11,702,703, and 11,773,447, each of which is incorporated by reference herein. Thus, typical portable and/or hand-held PCR arrays will include one or more buffers, nucleotides, and a polymerase. As noted above, there are numerous portable “lab-on-a-chip” devices known in the art (sec e.g., NantNudge device), and all of those are deemed suitable for use herein. However, it is further contemplated that in some cases, the PCR array may be directly integrated with the analytic device, while in other cases the PCR array may be informationally (e.g., via cable, Bluetooth, Wifi, etc.) coupled to such device. Regardless of the actual PCR array, it should be noted that a biological sample from the patient can be profiled on the hand-held portable PCR array by determining expression levels of clinically significant genes, wherein the PCR array comprises unique nucleic acid primers for evaluation of specific expression of multiple genes relevant to the first diagnosis. Finally, it should be recognized that the first diagnosis will typically also include reaction conditions for the PCR reaction (most typically time and temperature for denaturation, anneal/extension steps, number of cycles).

As also noted earlier, contemplated analytic devices will include a processor that is programmed to execute an algorithm that calculates the expression levels (and/or determines presence) of the clinically significant genes and that maps the expression levels of the genes to a genetically informed second diagnosis. Thus, based on the presence of certain genes and the expression levels of certain genes in conjunction with the first diagnosis, the processor will then map the expression levels (or presence of genes) to a genetically informed second diagnosis for the patient. Preferably, but not necessarily, mapping can be done via a reasoning engine, a via machine learning, and/or via probabilistic mapping and may use the same or a different processor. Viewed from a different perspective, the same point-of care device will not only allow receiving of initial digital diagnostic data and generation of a first diagnosis, but also be able to generate a genetically informed second diagnosis for the patient based on the PCR results from the (hand-held portable) PCR array. Advantageously, the initial diagnosis in such process is confirmed and even refined into a genetically informed second diagnosis.

Most typically, the confirmation and refinement will be performed on one or more processors (on board and/or remote) using algorithms as already noted above and described, for example, in U.S. Pat. Nos. 9,262,719, 9,530,100, 9,576,242, 10,255,552, 10,296,839, 10,296,840, and 10,762,433, each of which is incorporated by reference herein. Additionally, or alternatively, validation of the initial hypotheses can be performed using systems and methods as described in U.S. Pat. No. 10,354,194, and refinement (e.g., to assign a cancer score) can be performed using systems and methods described in US 2020/0335215, both incorporated by reference herein. Still further suitable algorithms, systems, and methods are described in US 2020/0356883 and US 2023/0034330, both incorporated by reference herein.

In yet further contemplated embodiments, the point-of-care analytic device may provide the genetically informed second diagnosis for the patient directly to a medical practitioner or to a computer device, which may be the same device or a networked computer, or a hand-held mobile device such as a tablet or mobile phone. Most typically, the analytic device will also generate a treatment plan at the point of care based on the second diagnosis to so guide a medical professional to select and administer a suitable pharmaceutical agent or treatment modality. As such, based on the PCR data obtained from the sample and analysis in the device, a drug such as an antibiotic or a chemotherapeutic drug can then be administered to the patient. Viewed from a different perspective, a medical professional only needs an analytical point-of-care device and a PCR array to generate actionable diagnostic information that can be used to treat a patient at the point of care

As such, and in view of the considerations provided above, the inventor also contemplates a computer-assisted method of translating polymerase chain reaction (PCR)-based diagnostic data into clinically actionable patient data. For example, digital PCR-based diagnostic data can be obtained from a PCR array as described above, and then processed in a processor via a probabilistic algorithm to establish a second genetically informed diagnosis. Where desired, this second diagnosis can then be further mapped via a processor using machine learning or artificial intelligence to generate a machine-learning informed third diagnosis or an artificial intelligence (AI) informed third diagnosis for the patient. Most typically, such machine-learning informed third diagnosis or an artificial intelligence (AI) informed third diagnosis is then conveyed to another computer device as noted above for guiding treatment of the patient. Refinement/mapping of the second diagnosis is typically performed by a reasoning engine or machine learning algorithm.

While it is generally contemplated that the systems and methods presented herein will be used for medical diagnosis and treatment of a human, it should also be appreciated that these methods are also suitable for analysis and processing of non-human subject data, following substantially the same methods as described above. Advantageously, and especially where the non-human subject is a companion animal (e.g., canine, feline, equine, etc.) or a livestock animal (e.g., porcine, bovine, avian, etc.), the tested condition need not only be limited to infections or cancers, or other serious diseases, but may also include various nutritional deficiencies such as macronutrient (e.g., protein, carbohydrate, lipid) deficiencies and micronutrient (e.g., vitamin or mineral) deficiencies.

Still further, and going beyond medical uses, it should be appreciated that the system and methods presented herein may also be employed in the context of environmental testing and remediation, and particularly suitable environmental uses include water testing (e.g., potable water, freshwater lakes and streams, pelagic, and marine bodies), soil testing (e.g., agricultural, horticultural), food and beverage testing, atmospheric testing, and especially where the test is concerned with a microbial or viral contaminant. For example, contemplated systems and methods may be readily used with a food or environmental sample to identify and characterize a pathogenic contaminant such as pathogenic bacteria, viruses, yeasts, fungi, microbes, and/or a parasites. Among other pathogens, the biological contaminant in food testing may be Salmonella spec. (e.g., for Salmonella enterica or Salmonella bongori serotypes) and PCR primers may thus be selected accordingly. As such tests will typically be more qualitative than quantitative, especially contemplated PCR assays will include Loop-mediated isothermal amplification (LAMP), Recombinase Polymerase Amplification (RPA), Recombinase Aided Amplification (RAA), Rolling Circle Amplification (RCA), or Saltatory Rolling Circle Amplification (SRCA). However, quantitative tests are also deemed suitable herein. Depending on the outcome of the analysis, it should be recognized that contemplated methods may therefore also include a step of applying a decontamination protocol to an object based on the second determination, wherein the sample was obtained from the object. Finally, and regardless of the type of test performed and medical or non-medical use, it is contemplated that the point-of-care or point-of-use will include in-patient and/or outpatient medical facilities, nursing homes, passenger airplanes, food preparation facilities (e.g., kitchen, a restaurant, or a cafeteria), and military installations.

It should be noted that any language directed to a computer should be read to include any suitable combination of computing devices, including servers, interfaces, systems, databases, agents, peers, engines, modules, controllers, or other types of computing devices operating individually or collectively. One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. In especially preferred embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges preferably are conducted over a packet-switched network, the Internet, LAN, WAN, VPN, or other type of packet switched network.

In some embodiments, the numbers expressing quantities of ingredients, properties such as concentration, reaction conditions, and so forth, used to describe and claim certain embodiments of the invention are to be understood as being modified in some instances by the term “about.” As used herein, the terms “about” and “approximately”, when referring to a specified, measurable value (such as a parameter, an amount, a temporal duration, and the like), is meant to encompass the specified value and variations of and from the specified value, such as variations of +/−10% or less, alternatively +/−5% or less, alternatively +/−1% or less, alternatively +/−0.1% or less of and from the specified value, insofar as such variations are appropriate to perform in the disclosed embodiments. Thus, the value to which the modifier “about” or “approximately” refers is itself also specifically disclosed. The recitation of ranges of values herein is merely intended to serve as a shorthand method of referring individually to each separate value falling within the range. Unless otherwise indicated herein, each individual value is incorporated into the specification as if it were individually recited herein.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). It should further be noted that the terms “prognosing” or “predicting” a condition, a susceptibility for development of a disease, or a response to an intended treatment is meant to cover the act of predicting or the prediction (but not treatment or diagnosis of) the condition, susceptibility and/or response, including the rate of progression, improvement, and/or duration of the condition in a subject.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

What is claimed is:

1. A computer assisted method of translating diagnostic data into polymerase chain reaction (PCR)-based clinically actionable patient data, the method comprising:
a) obtaining, via at least one processor, digital diagnostic data from a patient;
b) processing the digital diagnostic data via execution of an implementation of an algorithm, wherein the algorithm probabilistically maps the digital diagnostic data into a first diagnosis; and
c) profiling a biological sample from the patient on a hand-held portable PCR array by determining expression levels of clinically significant genes, wherein the PCR array comprises unique nucleic acid primers for evaluation of specific expression of multiple genes relevant to the first diagnosis;
d) mapping, via the at least one processor, the expression levels to a genetically informed second diagnosis for the patient; and
e) providing the genetically informed second diagnosis for the patient to a computer device.

2. The method of claim 1, wherein the first diagnosis relates to an infectious disease.

3. The method of claim 1, wherein the first diagnosis relates to resistance to treatment of an infectious disease.

4. The method of claim 1, wherein the first diagnosis relates to a cancer and/or comprises a determination of a cancer type, and optionally wherein the determination of the cancer type comprises a theranostic procedure.

5. The method of claim 1, wherein the digital diagnostic data is processed to arrive at the first diagnosis via a digital pathology platform.

6. The method of claim 1, wherein the algorithm comprises a machine learning algorithm.

7. The method of claim 1, wherein the algorithm comprises a reasoning algorithm executed by a reasoning engine.

8. The method of claim 1, wherein the digital diagnostic data comprise whole genomic or transcriptomic sequencing.

9. The method of claim 1, wherein the digital diagnostic data are derived from culturing a biological sample of the patient.

10. The method of claim 9, wherein the biological sample comprises a urine sample, blood sample, respiratory tract sample, mucosal sample, or tissue biopsy.

11. The method of claim 1, wherein the digital diagnostic data are derived from a radiological image.

12. The method of claim 1, further comprising generating a treatment plan at a point of care based on the second diagnosis.

13. The method of claim 12, further comprising administration of a drug based on the treatment plan, and wherein the drug is an antibiotic or a chemotherapeutic drug.

14. A computer assisted method of translating water or food testing data into polymerase chain reaction (PCR)-based actionable data, the method comprising:
a) obtaining, via at least one processor, digital data from an assay selected from the group consisting of a food quality assay, a beverage quality assay, and a water quality assay, wherein the assay determines a presence and/or a level of at least one biological contaminant in a sample, and wherein the assay is performed on one portion of the sample;
b) processing the digital data via execution of an implementation of an algorithm, wherein the algorithm probabilistically maps the data into a first determination of biological contamination;
c) profiling another portion of the sample on a hand-held portable PCR array to determine expression levels of genes related to microbial presence and quantity, wherein the PCR array comprises multiple unique nucleic acid primers for evaluation of expression of respective multiple genes relevant to the first determination;
d) mapping, via the at least one processor, the gene expression levels to a genetically informed and actionable second determination; and
e) providing the genetically informed and actionable second determination of biological contamination to a computer device.

15. The method of claim 14, wherein the biological contaminant comprises a bacterium, a virus, a yeast, a fungus, a microbe, or a parasite.

16. The method of claim 15, wherein the assay comprises Loop-mediated isothermal amplification (LAMP), Recombinase Polymerase Amplification (RPA), Recombinase Aided Amplification (RAA), Rolling Circle Amplification (RCA), or Saltatory Rolling Circle Amplification (SRCA).

17. The method of claim 14, wherein the sample is obtained from an in-patient facility, an outpatient medical facility, a nursing home, a cruise ship, a passenger airplane, or a food preparation facility.

18. The method of claim 14, further comprising a step of applying a decontamination protocol to an object based on the second determination, wherein the sample was obtained from the object.

19. A computer assisted method of translating diagnostic data into polymerase chain reaction (PCR)-based actionable non-human subject data, the method comprising:
a) obtaining, via at least one processor, digital diagnostic data from a non-human subject;
b) processing the digital diagnostic data via execution of an implementation of an algorithm, wherein the algorithm probabilistically maps the digital diagnostic data into a first diagnosis; and
c) profiling a biological sample from the non-human subject on a hand-held portable PCR array by determining expression levels of nutritionally significant genes, wherein the PCR array comprises multiple unique nucleic acid primers for evaluation of expression of respective multiple genes relevant to the first diagnosis;
d) mapping, via the at least one processor, the expression levels to a genetically informed second diagnosis for the subject; and
e) providing the genetically informed second diagnosis for the subject to a computer device.

20. The method of claim 19, wherein the first diagnosis relates to a state of nutritional deficiency in the subject, and wherein the nutritional deficiency is a macronutrient deficiency or micronutrient deficiency.
